(19) United States
(12) Patent Application Publication
(10) Pub. No.: US 2025/0090303 A1
(43) Pub. Date: Mar. 20, 2025

(54) ARTIFICIAL LENS SAC FOR FIXING ARTIFICIAL LENS FOR CATARACT SURGERY

(71) Applicants: Industry-Academic Cooperation Foundation Gyeonsang National University, Jinju-si (KR); Korea University Research and Business Foundation, Seoul (KR)

(72) Inventors: Seong-Jae KIM, Jinju-si (KR); Youngsub EOM, Seoul (KR)

(73) Assignees: Industry-Academic Cooperation Foundation Gyeonsang National University, Jinju-si (KR); Korea University Research and Business Foundation, Seoul (KR)

(21) Appl. No.: 18/468,255

(22) Filed: Sep. 15, 2023

(51) Int. Cl. A61F 2/16; A61F 2/00

(52) U.S. Cl. CPC A61F 2/16; A61F 2002/0081; A61F 2002/169; A61F 2002/16902; A61F 2210/0057; A61F 2220/0008

(57) ABSTRACT

The present disclosure provides an artificial lens fixing sac for fixing an artificial lens for cataract surgery. The artificial lens fixing sac for fixing an artificial lens for cataract surgery of the present disclosure is installed at a position of a lens that is removed by cataract surgery, and the artificial lens fixing sac includes an implantation hole in which an artificial lens, which is inserted into an internal space of the artificial lens fixing sac, is implanted at the position of the removed lens, and fixing threads installed at predetermined intervals on an outer peripheral edge of the artificial lens fixing sac to fix the artificial lens fixing sac to a sclera.

### TECHNICAL FIELD

The present disclosure relates to an artificial lens fixing sac, which is a device for placing an artificial lens in a patient who does not have a lenticular capsule or has an unstable lenticular capsule into which an artificial lens may be inserted during surgery for treating cataract, the artificial lens fixing sac being capable of preventing the artificial lens from moving into an iris or a vitreous body while reducing an injury to a cornea or a risk of an increase in intraocular pressure.

### BACKGROUND

In general, cataract is a disease in which the inside of a lens becomes cloudy, which causes objects to appear hazy and foggy.

The cataract is treated with surgery, and the treatment of cataract is performed in the following steps.

First, a part of a transparent cornea is incised, a lenticular capsule positioned inside the transparent cornea is incised, contents in a cloudy lens are removed by phacoemulsification, and an artificial lens is inserted into the lenticular capsule from which the contents are removed.

However, the general artificial lens implantation cannot be performed sometimes.

In the first case of an eye with no lens in which a lenticular capsule is not supported or in the second case in which an artificial lens is separated, secondary artificial lens implantation needs to be performed because a lenticular capsule is damaged or there is no lenticular capsule in an eyeball into which a lens is to be inserted.

There are broadly two types of secondary artificial lens implantation, in which the first type is sutured posterior artificial lens implantation, and the second type is anterior artificial lens implantation.

The sutured posterior artificial lens implantation is performed by various methods. The methods are commonly performed by tying a thread to a support portion (haptic) of the artificial lens to fix the artificial lens to a ciliary sulcus.

Various types of complications may occur after the sutured posterior artificial lens implantation. The artificial lens fixed to the ciliary sulcus deviates from a center and is tilted toward one side, or the eyesight is not as good as expected after the procedure because of corneal astigmatism.

Recently, non-sutured posterior artificial lens fixation using a sclera tunnel has been reported to somewhat solve the above-mentioned problem. However, the non-sutured posterior artificial lens fixation is more complicated and requires a larger amount of time because of the surgical technique that incises the sclera. Further, the non-sutured posterior artificial lens fixation causes a great risk of infection after the procedure.

As the related art related to the present disclosure, Patent No. 10-1555298 (hereinafter, referred to as the ‘patent document’) registered with the Korean Intellectual Property Office has been proposed.

The patent document provides a configuration including: a body disposed in a lenticular capsule and having a partially opened ring shape to allow the lenticular capsule to be kept in a preset shape; and protruding portions formed at preset intervals on the body and protruding from the body by a first preset length toward a posterior lenticular capsule and then protruding by a preset length into the body, the protruding portions being configured to support an artificial lens in the lenticular capsule and prevent the artificial lens from moving toward the posterior lenticular capsule and made of a material that is stretched by a force with preset strength or higher from the outside to minimize an incision width of the cornea.

The configuration of the patent document is disposed in the lens to maintain the shape of the lenticular capsule and fixes the position of the artificial lens without being affected by partial damage to an eyeball such as the lenticular capsule, but the configuration of the patent document does not cause complications.

However, because the configuration of the patent document has the structure in which the protruding portions protrude from the body and are disposed at predetermined intervals, it is not easy to handle the configuration of the patent document. Further, spaces present between the protruding portions may cause a problem in which a haptic part of the artificial lens is not easily fixed.

### DOCUMENT OF RELATED ART

### Patent Document

(Patent Document 1) Korean Patent No. 10-1555298 (registered on Sep. 17, 2015)

### SUMMARY

The present disclosure has been made in an effort to insert, by using an injector, an artificial lens to be inserted to be substituted for a lens that is removed during cataract surgery.

The present disclosure has also been made in an effort to stably fix and place an artificial lens in an internal space of an artificial lens fixing sac by being guided and fixed by a haptic part.

The present disclosure has also been made in an effort to stably fix an artificial lens to minimize complications such as damage to a cornea, an increase in intraocular pressure, and a positional abnormality of an artificial lens.

An exemplary embodiment of the present disclosure provides an artificial lens fixing sac for fixing an artificial lens for cataract surgery, which is installed at a position of a lens that is removed by cataract surgery, the artificial lens fixing sac including: an implantation hole in which an artificial lens, which is inserted into an internal space of the artificial lens fixing sac, is implanted; and fixing threads installed at predetermined intervals on an outer peripheral edge of the artificial lens fixing sac to fix the artificial lens fixing sac to a sclera.

In the configuration for achieving the objects of the present disclosure, the artificial lens fixing sac may have a shape in which circumferential surfaces of first and second plates having corresponding shapes are in contact with each other, and the internal space into which the artificial lens is inserted may be formed in the shape in which the first and second plates are in contact with each other.

In the configuration for achieving the objects of the present disclosure, the first and second plates of the artificial lens fixing sac may have corresponding shapes and inner surfaces each having a concave disk shape, and an implantation hole may be formed at a center of the first and second plates to prevent an increase in intraocular pressure and ensure a flow of water.

In the configuration for achieving the objects of the present disclosure, one or more marking holes may be formed in the first and second plates of the artificial lens fixing sac to set directionality at the time of fixing the artificial lens fixing sac between the implantation hole and the circumferential surface.

In the configuration for achieving the objects of the present disclosure, the internal space, which is defined as inner surfaces of the first and second plates of the artificial lens fixing sac, which are each formed in a concave disk shape, face each other, may include a guide surface formed to guide a haptic part of the artificial lens and defined as a width of the internal space gradually decreases toward the circumferential surface, and the circumferential surfaces, which are in contact with each other while defining a curved surface, may include a fixing groove having a valley shape and formed to fix and support the haptic part of the artificial lens.

In the configuration for achieving the objects of the present disclosure, the artificial lens fixing sac may be made of a 20d silicone material so that the artificial lens fixing sac is freely folded or unfolded when the artificial lens fixing sac is mounted in an iris.

In the configuration for achieving the objects of the present disclosure, the implantation hole may be formed at the center of the first and second plates so as to have a diameter of the artificial lens, marking holes may be formed at predetermined intervals around the implantation hole, and the marking hole may be formed in any one of circular, triangular, quadrangular, and hexagonal shapes.

In the configuration for achieving the objects of the present disclosure, the marking holes may be formed at the predetermined intervals around the implantation hole, and one side of each of the marking holes may be cut out toward the implantation hole.

According to the present disclosure, it is possible to insert, by using the injector, the artificial lens to be inserted to be substituted for a lens that is removed during cataract surgery.

According to the present disclosure, it is possible to stably fix and place the artificial lens in the internal space of the artificial lens fixing sac by being guided and fixed by the haptic part.

According to the present disclosure, it is possible to stably fix the artificial lens to minimize complications such as damage to a cornea, an increase in intraocular pressure, and a positional abnormality of the artificial lens.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 is a perspective view of an artificial lens fixing sac to which the present disclosure is applied.

FIG. 2 is a front state view of the artificial lens fixing sac to which the present disclosure is applied.

FIG. 3 is a top-plan state view of FIG. 2.

FIG. 4 is a longitudinal sectional state view of FIG. 2.

FIG. 5 is a state view in which a fixing thread is connected to the artificial lens fixing sac to which the present disclosure is applied.

FIG. 6 is a state view illustrating a state in which an artificial lens is mounted in the artificial lens fixing sac in FIG. 5.

FIG. 7 is a cross-sectional state view illustrating a state in which the artificial lens is implanted in an eyeball.

FIG. 8 is a view illustrating one embodiment of the present disclosure.

### DETAILED DESCRIPTION

In the following detailed description, reference is made to the accompanying drawing, which forms a part hereof. The illustrative embodiments described in the detailed description, drawing, and claims are not meant to be limiting. Other embodiments may be utilized, and other changes may be made, without departing from the spirit or scope of the subject matter presented here.

Particular exemplary embodiments for carrying out the present disclosure will be described with reference to the accompanying drawings.

These embodiments are described in detail sufficient to enable those skilled in the art to carry out the present disclosure, and it should be understood that the various embodiments of the present disclosure are different but need not be mutually exclusive.

Hereinafter, the exemplary embodiments of the present disclosure will be described in detail with reference to the accompanying drawings so that those skilled in the art to which the present disclosure pertains may easily carry out the present disclosure.

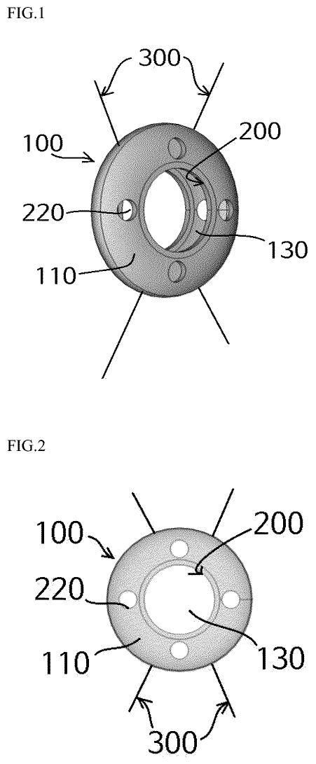

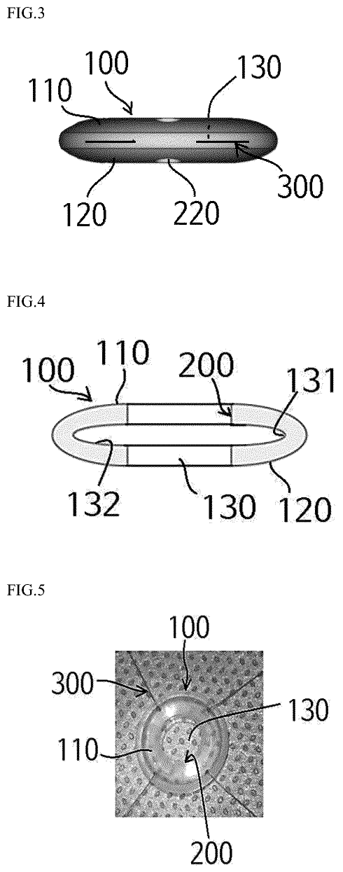

FIG. 1 is a perspective view of an artificial lens fixing sac to which the present disclosure is applied, FIG. 2 is a front state view of the artificial lens fixing sac to which the present disclosure is applied, FIG. 3 is a top-plan state view of FIG. 2, and FIG. 4 is a longitudinal sectional state view of FIG. 2.

An artificial lens fixing sac 100 for fixing an artificial lens for cataract surgery to which the present disclosure is applied has a structure that allows an artificial lens 400, which is selected to be substituted for a lens removed by cataract surgery, to be stably mounted at the parts of the lens.

The artificial lens fixing sac 100 needs to be unfolded by restoration characteristics made by material properties after the artificial lens fixing sac 100 is folded and inserted during the surgery. Therefore, the artificial lens fixing sac 100 may be made of a silicone material, particularly, a 20d silicone material.

The artificial lens fixing sac 100 may be formed in a disk shape because the artificial lens fixing sac 100 is fixed in an iris. The artificial lens fixing sac 100 has an internal space 130 through which the artificial lens 400 is inserted into the artificial lens fixing sac 100 in a state in which the artificial lens fixing sac 100 is fixed to the iris.

The artificial lens fixing sac 100 is divided into a first plate 110 and a second plate 120, inner surfaces of the first and second plates 110 and 120 each having a disk shape are concavely formed, and the first and second plates 110 and 120 are bonded and fixed in a state in which circumferential surfaces of the first and second plates 110 and 120 are in contact with each other, such that the internal space 130 may be formed in the artificial lens fixing sac 100.

In addition to the above-mentioned method of bonding the circumferential surfaces of the first and second plates 110 and 120, the internal space 130 may also be formed between the first and second plates 110 and 120 of the artificial lens fixing sac 100 by injecting liquid silicone into a mold with high pressure.

An implantation hole 200 is formed by perforating a central position of the first and second plates 110 and 120 to stably mount the artificial lens 400 inserted into the artificial lens fixing sac 100, prevent an increase in intraocular pressure, and ensure a flow of water.

The implantation hole 200 formed at the central position of the first and second plates 110 and 120 surrounds an outer peripheral edge of the artificial lens 400 inserted into the internal space 130 with an inner peripheral edge of the implantation hole 200. Therefore, the artificial lens 400 is quickly positioned in the first and second plates 110 and 120, a stable mounted state may be maintained after the artificial lens 400 is positioned, an increase in intraocular pressure is prevented, and a smooth flow of water is ensured.

One or more marking holes 220 are formed in the first and second plates 110 and 120 to set the directionality at the time of fixing the artificial lens fixing sac between the implantation hole 200, the first and second plates 110 and 120, and the circumferential surface.

FIG. 5 is a state view in which a fixing thread is connected to the artificial lens fixing sac to which the present disclosure is applied.

The marking holes 220 are formed by perforating four to eight peripheral portions around the implantation hole 200 of the artificial lens fixing sac 100 at predetermined intervals.

The plurality of marking holes 220 serve to allow a practitioner to find a sclera fixing position of fixing threads 300 at the time of fixing the artificial lens fixing sac 100 to the iris, thereby helping to expedite the surgery and providing the patient with stable eyesight after the surgery.

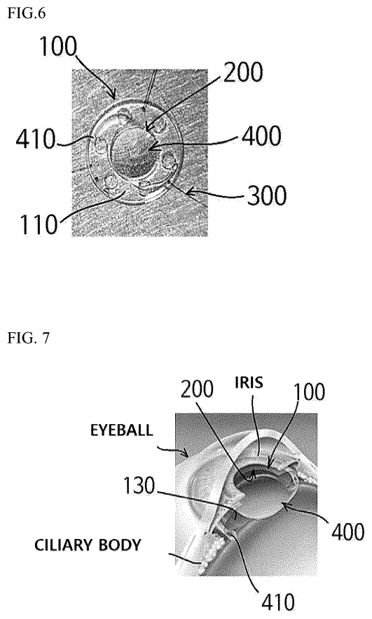

FIG. 6 is a state view illustrating a state in which an artificial lens is mounted in the artificial lens fixing sac in FIG. 5, and FIG. 7 is a cross-sectional state view illustrating a state in which the artificial lens is implanted in an eyeball. As illustrated in FIGS. 6 and 4, in the implantation hole 200 formed at the center of the internal space 130 of the artificial lens fixing sac 100, a guide surface 132 is defined as a width of the internal space 130 decreases toward the circumferential surfaces of the first and second plates 110 and 120. The circumferential surfaces of the first and second plates 110 and 120 are in contact with each other while defining a curved surface, such that a fixing groove 131 is defined. A haptic part 410 of the artificial lens 400, which is inserted into the internal space 130, naturally moves along a gradient of the guide surface 132 and reaches the fixing groove 131 having a valley shape. The stable fixed state of the haptic part 410, which has reached the fixing groove 131, is maintained by the valley shape without a separate fixing force.

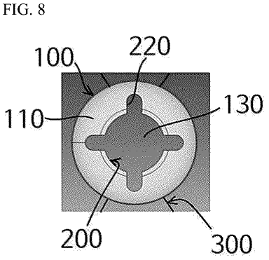

FIG. 8 is a view illustrating a state of one embodiment of the present disclosure. In the present disclosure, the fixing thread 300 bound to an outer peripheral edge of the artificial lens fixing sac 100 is used to fix the artificial lens fixing sac 100, which has the internal space 130 into which the artificial lens 400 is inserted, to the iris. First, the practitioner moves the artificial lens fixing sac 100 to the fixing position on the iris and finely adjusts the position of the artificial lens depending on his/her skill. Then, the practitioner inserts the fixing thread 300 into the iris and then fixes the fixing thread by melting a surgical thread by using a surgical tool.

In this case, it is not easy to identify whether the artificial lens fixing sac 100 is accurately installed at the position at which the artificial lens fixing sac 100 is to be installed.

The practitioner may easily identify whether the artificial lens fixing sac 100 is installed at the accurate position by using the marking holes 220 formed in the several peripheral portions around the implantation hole 200.

That is, the installation position of the artificial lens fixing sac is set by adjusting the position of the artificial lens fixing sac 100 while fixing one fixing thread 300, which is bound to the outer peripheral edge of the artificial lens fixing sac 100, to the rear side of the iris and fixing another fixing thread 300 to the rear side of the iris.

As the operation of fixing the fixing threads 300 is repeated, the artificial lens fixing sac 100 is installed at the accurate position on the iris and fixed without moving by the operation of melting the fixing thread 300.

The process of recognizing the state of the artificial lens fixing sac 100 while fixing the fixing thread 300 to the rear side of the iris and the process of determining whether the artificial lens fixing sac 100 is installed at the accurate position may be performed by the practitioner by visually comparing and identifying the horizontal and symmetric states of the marking holes 220 formed in the several portions.

Therefore, the marking hole 220 may be formed in any one of circular, quadrangular, triangular, and octagonal shapes. When the marking hole 220 may be formed in a circular shape in which one side thereof is cut out toward the implantation hole 200.

In addition, the marking holes 220 are formed by perforating four to eight peripheral portions around the implantation hole 200. The practitioner meets the accuracy by adjusting the position of the artificial lens fixing sac 100 by comparing and determining the positions of the marking holes 220 formed during the process of installing the artificial lens fixing sac 100. Therefore, the artificial lens fixing sac 100 may be installed in the accurate state at the position on the iris.

While the exemplary embodiments of the present disclosure have been described above with reference to the accompanying drawings, the present disclosure is not limited to the exemplary embodiments disclosed above but will be implemented in various different forms, and those skilled in the art will understand that the present disclosure may be implemented in any other specific form without changing the technical spirit or the essential feature of the present disclosure.

Therefore, it should be understood that the above-described embodiments are illustrative in all aspects and do not limit the present specification.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

What is claimed is:

1. An artificial lens fixing sac for fixing an artificial lens for cataract surgery, which is installed at a position of a lens that is removed by cataract surgery, the artificial lens fixing sac comprising:
an implantation hole in which an artificial lens, which is inserted into an internal space of the artificial lens fixing sac, is implanted; and
fixing threads installed at predetermined intervals on an outer peripheral edge of the artificial lens fixing sac to fix the artificial lens fixing sac to a sclera.

2. The artificial lens fixing sac of claim 1, wherein the artificial lens fixing sac has a shape in which circumferential surfaces of first and second plates having corresponding shapes are in contact with each other, and the internal space into which the artificial lens is inserted is formed in the shape in which the first and second plates are in contact with each other.

3. The artificial lens fixing sac of claim 2, wherein the first and second plates of the artificial lens fixing sac have corresponding shapes and inner surfaces each having a concave disk shape, and an implantation hole is formed at a center of the first and second plates to prevent an increase in intraocular pressure and ensure a flow of water.

4. The artificial lens fixing sac of claim 3, wherein one or more marking holes are formed in the first and second plates of the artificial lens fixing sac to set directionality at the time of fixing the artificial lens fixing sac between the implantation hole and the circumferential surface.

5. The artificial lens fixing sac of claim 2, wherein the internal space, which is defined as inner surfaces of the first and second plates of the artificial lens fixing sac, which are each formed in a concave disk shape, face each other, includes a guide surface formed to guide a haptic part of the artificial lens and defined as a width of the internal space gradually decreases toward the circumferential surface, and
wherein the circumferential surfaces, which are in contact with each other while defining a curved surface, includes a fixing groove having a valley shape and formed to fix and support the haptic part of the artificial lens.

6. The artificial lens fixing sac of claim 1, wherein the artificial lens fixing sac is made of a 20d silicone material so that the artificial lens fixing sac is freely folded or unfolded when the artificial lens fixing sac is mounted in an iris.

7. The artificial lens fixing sac of claim 3, wherein the implantation hole is formed at the center of the first and second plates so as to have a diameter of the artificial lens, marking holes are formed at predetermined intervals around the implantation hole, and the marking hole is formed in any one of circular, triangular, quadrangular, and hexagonal shapes.

8. The artificial lens fixing sac of claim 4, wherein the marking holes are formed at the predetermined intervals around the implantation hole, and one side of each of the marking holes is cut out toward the implantation hole.
